(19) United States
(12) Patent Application Publication
(10) Pub. No.: US 2025/0319132 A1
(43) Pub. Date: Oct. 16, 2025

(54) Methods and compositions for improving the immune response against viral pathogens

(71) Applicant: Mirror Biologics, Inc., Wesley Chapel, FL (US)

(72) Inventor: Michael Har-Noy, Wesley Chapel, FL (US)

(21) Appl. No.: 19/180,751

(22) Filed: Apr. 16, 2025

Related U.S. Application Data
(60) Provisional application No. 63/634,809, filed on Apr. 16, 2024.

(51) Int. Cl. A61K 35/17; A61K 40/11; A61K 40/41; A61K 40/42; A61P 37/04; C07K 14/525; C07K 14/535; C07K 14/57; C07K 14/705

(52) U.S. Cl. CPC A61K 35/17; A61K 40/11; A61K 40/418; A61K 40/421; A61P 37/04; C07K 14/525; C07K 14/535; C07K 14/57; C07K 14/70575; A61K 2239/31; A61K 2239/38

(57) ABSTRACT

Immunotherapy regimens against a viral pathogen in individuals are disclosed. The immunotherapy regimen is a universal vaccine that is administered intradermally. Multiple intradermal doses of the universal vaccine are administered to elderly individuals to prime the individual's immune system for an effective response against a viral pathogen.

### FIELD

The disclosure relates to immunotherapy against infections and more particularly relates to vaccines against viral pathogens.

### BACKGROUND

Harnessing the power of the immune system to treat infectious diseases is a major goal of immunotherapy. Vaccination (a/k/a, active immunotherapy) methods are designed to activate the immune system to specifically recognize and protect against invading pathogens. For over 200 years, active immunotherapy approaches have been used to prevent numerous infectious diseases, including smallpox, rabies, typhoid, cholera, plague, measles, varicella, mumps, poliomyelitis, hepatitis B, and the tetanus and diphtheria toxins.

Active immunotherapy concepts are now being applied to develop preventive or therapeutic vaccines against viral infections. However, existing immunotherapy technology has not been successful in protecting against many of the modern disease targets such as hepatitis C, HIV/AIDS, Dengue, herpes viruses, cytomegalovirus, and emerging viral threats such as coronaviruses including SARS, MERS and COVID19. This is in part due to the inability of current vaccination technology to elicit the correct type of immune responses.

### Definitions

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers to one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably and have the same meaning that includes a single element and a plurality of the elements and may also be represented by the suffix “(s)” at the end of the element.

The terms “about” and “substantially” are used herein with respect to measurable values and ranges due to expected variations known to those skilled in the art (e.g., limitations and variability in measurements).

The term “and/or” means one or all the listed elements or a combination of any two or more of the listed elements.

The terms “comprises,” “comprising,” and variations thereof are to be construed as open ended—i.e., additional elements or steps are optional and may or may not be present.

“Vaccine composition” as referred to herein relates to a composition that is preventive and/or therapeutic against an invading pathogen.

“Compositions” as referred to herein relate to preventive and/or therapeutic compositions.

“Universal vaccine” as referred to herein relates to a vaccine composition that is used to prime a host susceptible to pathogenic infection. The universal vaccine does not engender a pathogen specific response but provides a non-specific immune response upon infection by both known and unknown pathogens that is capable of suppressing and eventually eliminating the pathogen.

“Host” as referred to herein relates to an individual that is administered the universal vaccine compositions described herein. The host can be an individual that is susceptible to an infectious disease and may also be referred to as the susceptible individual.

“Immunocompromised” or “immunosuppressed” as referred to herein relates to having a weakened immune system. Individuals who are immunocompromised or immunosuppressed have a reduced ability to fight infections and other diseases. This reduced ability may be due the senescence of the immune system's ability to mount a cellular immune response as a consequence of aging. This may also be caused by certain diseases or conditions, such as AIDS, cancer, diabetes, malnutrition, and certain genetic disorders. It may also be caused by certain medicines or treatments, such as anticancer drugs, radiation therapy, and stem cell or organ transplant.

“Immunotherapy regimen” or “regimen” as referred to herein relates to a method of administering the living allogeneic immune cell therapy described herein either alone or in combination with specific antigens. The regimen can include multiple doses of allogeneic immune cell therapy with or without specific antigens as a vaccine composition.

“Priming” as referred to herein relates to modulating a host's immune system to improve the ability of the host's immune system to mount an effective response upon infection with an invading pathogen.

“Progressive viral infection” as referred to herein relates to an infection by a virus that can cause tissue damage and adversely impacts one or more organ functions in the infected individual as the viral load becomes greater, such as lungs/respiratory tract, GI tract, liver and kidneys where an adequate immune response is either not present to reduce and/or eliminate the viral load or the immune response is late, incorrect and/or over-activated and dys-regulated.

“Universal vaccine” as referred to herein relates to a composition that improves the ability of the host's immune system to mount an effective response against an invading pathogen. The universal vaccine does not necessarily educate the immune system in advance to the specific viral antigens.

“Pathogens” as referred to herein relates to disease causing entities and can include bacteria, viruses, parasites and the like.

“Viral pathogens” as referred to herein relates to viral disease causing entities including, but not limited to, noroviruses, rotaviruses, adenoviruses, astroviruses, herpes viruses, retroviruses, flavivirid viruses, picoviruses, polyoma viruses, toroviruses, coronaviruses (including SARS, MERS and SARS-COV-2), exanthematous poxviruses (chickenpox, smallpox, varicella-zoster), hemorrhagic viruses such as viruses that cause ebola, Lassa Fever, dengue fever, yellow fever, Marburg and Crimean-Congo; neurological viruses such as polio, viral meningitis, viral encephalitis and rabies.

“COVID-19” as referred to herein relates to the disease in an individual resulting from infection by SARS-COV-2 virus.

“Elderly individuals” as referred to herein relates to individuals affected by immunosenescence, a natural loss in individuals of the ability to mount an effective cellular immune response to invading pathogens. In one embodiment, such an individual is over the age of 50 years, or over the age of 60 years, or over the age of 70 years.

### SUMMARY

A method of improving the immune response in an individual against a viral pathogen comprises administering allogeneic living immune cells where at least a portion are T-cells wherein the T-cells comprise effector/memory T-cells of a Th1 phenotype that produces IFN-γ and not IL-4.

In a further embodiment the method comprises the allogeneic living immune cells being administered in at least three intradermal doses.

In a further embodiment wherein, the method comprises the effector/memory T-cells express CD45RO+ and CD62LLo.

In a further embodiment wherein, the method comprises the effector/memory Th1 cells are activated at formulation or introduction to the individual.

In a further embodiment the method comprises the activation of the T-cells which comprises cross-linking of CD3 and CD28 surface molecules on the T-cells.

In a further embodiment the method comprises the T-cells cells that express CD40L upon being activated and produce inflammatory cytokines.

In a further embodiment the method comprises wherein the inflammatory cytokines comprise IFN-γ, GM-CSF, or TNF-α or combinations thereof.

In a further embodiment the method comprises wherein at least a portion of the effector/memory T-cells are derived from naïve CD4+ T-cells from a donor other than the individual.

In a further embodiment wherein each of the doses of intradermal doses are administered at an interval of about 3 to 4 days.

In a further embodiment wherein, the invading pathogen is a virus.

In a further embodiment wherein, the invading pathogen is a coronavirus, RSV, Influenza A or Influenza B.

In a further embodiment wherein, the individual is over the age of 60.

In a further embodiment wherein administration of the allogeneic living immune cells increases the level of IFN-gamma in the individual and/or the Th1 response after pathogen infection.

In a further embodiment wherein the method comprises Th1/Th2 balance shifts toward Th1.

In a further embodiment wherein, the method comprises a fourth, a fifth, or more intradermal doses are administered to the individual.

In a further embodiment wherein, the method comprises the allogeneic living immune cells being administered with selected viral antigens.

In yet another embodiment a method is described of modulating the immune response in an individual at least 60 years of age comprising administering allogeneic living immune cells where at least a portion are T-cells wherein the T-cells comprise effector/memory T-cells of a Th1 phenotype that increases the Th1/Th2 ratio.

In a further embodiment the method comprises allogeneic living immune cells being administered in at least three intradermal doses.

In a further embodiment, the method comprises the effector/memory T-cells expressing CD45RO+ and CD62LLo.

In a further embodiment wherein, the method comprises the effector/memory Th1 cells being activated at formulation or introduction to the individual.

In a further embodiment the method comprises the activation of the T-cells comprises cross-linking of CD3 and CD28 surface molecules on the T-cells.

In a further embodiment wherein, the method comprises the T-cells cells expressing CD40L upon being activated and produce inflammatory cytokines.

In a further embodiment the method comprises the inflammatory cytokines comprising IFN-γ, GM-CSF, or TNF-α or combinations thereof.

In a further embodiment the method comprises at least a portion of the effector/memory T-cells being derived from naïve CD4+ T-cells from a donor other than the individual.

In a further embodiment the method comprises each of the doses of intradermal doses being administered at an interval of about 3 to 4 days.

In a further embodiment the method comprises administration of the allogeneic living immune cells increasing the level of IFN-gamma in the individual and/or the Th1 response after pathogen infection.

In a further embodiment the method comprises a fourth, a fifth, or more intradermal doses being administered to the individual.

In a further embodiment the method comprises the allogeneic living immune cells being administered with selected viral antigens.

Another aspect of the present disclosure relates to a method of increasing vaccine responsiveness in an individual fully vaccinated against at least one viral infection without additional doses of a vaccine for the at least one viral infection and comprising administering one or more doses of a vaccine composition to the fully vaccinated individual, the vaccine composition comprising allogeneic effector/memory Th-1 cells derived from a deliberately HLA-mismatched donor to the individual.

In one or more embodiments, the method further comprises administering at least three doses of the vaccine composition.

In one or more embodiments, the at least three doses of the vaccine composition may be administered by the same route or different routes at each administration.

At least a portion of the effector/memory T-cells are derived from naïve CD4+ T-cells from a donor other than the individual.

The effector/memory T-cells express CD45RO+ and CD62L^{Lo}.

Another aspect of the present disclosure relates to a kit for preventing progression of viral infections. The kit comprises multiple doses of a vaccine composition-referred to as vaccine dose. A first vaccine dose comprises allogeneic living immune cells where at least a portion are T-cells wherein the T-cells comprise effector/memory T-cells of a Th1 phenotype that produces IFN-γ and not IL-4 wherein the allogenic living immune cells are provided at a first concentration; a second vaccine dose comprising the allogenic living immune cells provided in a second concentration; and a third vaccine dose comprising the allogenic living immune cells provided in a third concentration.

In one or more embodiments, the first concentration, second concentration and third concentration are each within the range of about 1×10⁷ to about 1×10⁹ cells/mL and in one or more embodiments one or more of the doses is approximately 0.5 mL.

In one more embodiments, the first vaccine dose, second vaccine dose, and third vaccine dose are the same or different in concentration and/or volume.

In one or more embodiments, the effector/memory T-cells express CD45RO+ and CD62LLo.

In one or more embodiments, the kit further comprises a fourth vaccine dose comprising the allogenic living immune cells provided in a fourth concentration wherein the fourth concentration is the same as the first, second and/or third concentration.

In one or more embodiments, the kit further comprises a fifth vaccine dose comprising the allogenic living immune cells provided in a fifth concentration wherein the fifth concentration is the same as the first, second, third and/or fourth concentration.

In one or more embodiments, the allogenic living immune cells comprise T-cells that can express CD40L upon being activated and produce inflammatory cytokines.

### BRIEF DESCRIPTION OF THE DRAWINGS

FIG. 1 is a schematic representation of the mechanism of action of this disclosure.

FIG. 2 is a series of graphical views of PBMC isolated from Day 0, 10, 28, 168 and 336 incubated with cell activation cocktails: Phorbol myristate acetate (PMA), Alloantigen a lysate from AlloStim® cells and Resiquimod (R848).

FIGS. 3, 4, 5 and 6 are graphical views of supernatants from the activated PBMC co-incubated with live viral-infected cell line cultures RSV, Human Coronavirus, Influenza A, and Influenza B virus.

### DETAILED DESCRIPTION OF ILLUSTRATIVE EMBODIMENTS

The present disclosure includes novel approaches to addressing the problem of emerging viral epidemics, especially in vulnerable elderly or immunosuppressed individuals. The present disclosure includes a novel universal vaccine to protect individuals from progressive viral infections. The universal vaccine can include the use of allogeneic living immune cells, for example, living Th1-like memory cells in an activated state. The immune cells are administered in an immunotherapy regimen to modulate the immune systems of vulnerable elderly or immunosuppressed individuals in a manner that primes the individuals to respond quickly to any viral infection. The immunotherapy described herein is particularly advantageous for elderly or immunosuppressed individuals because it can improve the immune response of the individual by overcoming the late innate response and cellular immune suppression common in the elderly immune response or in the immunosuppressed to viral infections.

The novel universal vaccine can protect elderly, immunocompromised or immunosuppressed, and/or healthy individuals from any type of progressive viral infection, including, for example, protection against different strains of influenza (both known and unknown), protection from coronaviruses, e.g., SARS-COV-2 infection, and any future variants or mutations and any currently unknown future emergent novel viruses. The protection mechanism can be designed to prime the immune system of the elderly or the immunosuppressed to respond rapidly to a viral infection and confer protection through rapid viral clearance and in-situ vaccination to elicit a memory immune response to protect against recurrence.

The priming can be non-specific to the type of viral infection and thus serves as a “universal vaccine”. As a universal vaccine, it is not necessary to know and educate the immune system in advance to the specific viral antigens. Advantageously, applied to the broad population, the rapid clearance of virus can reduce virulence and person-to-person spread preventing pandemic emergencies.

In one or more embodiments, the priming can be specific to the type of viral infection. The universal vaccine can be administered with internal viral proteins either in the original formulation or separately. The internal viral proteins may increase the depth and breadth of the cellular immune repertoire in an individual and/or accelerate the process. Specifically, internal viral proteins may be administered with the universal vaccine to simulate environmental exposures over time. The internal viral proteins could be conjugated to biodegradable beads.

Limiting the extent of viral spread in host tissues can be achieved by a simulated rapid innate immune response through the bystander activation of allo-specific Th1 cells in circulation resulting from the intradermal dosing of allogeneic cells. The simulated innate immune response can control viral burden while providing sufficient time, and creating the immunological conditions necessary, for development of specific adaptive immune responses to the invading virus, such as cellular immune responses. The establishment of an effective specific cellular immune response can lead to rapid recovery from viral infection and resistance to re-infection.

The aging of the immune system loses Th1 immunity resulting in a lower Th1/Th2 balance, which results in weakened and/or delayed cellular immune responses. Further, chronic inflammation, such as chronic CMV infection in the elderly can result in exhaustion of memory cells. The loss of thymic function in the elderly can limit the T-cell repertoire. These immune defects can make elderly individuals more susceptible to the severe consequences of viral infections. Elderly adults can be vulnerable to severe morbidity and mortality associated with respiratory viral infection. The aged immune system can be deficient in the ability to mount an effective cellular immune response and delayed immune responses of weak immune systems of the elderly can often result in high viral burdens which often leads to immune over-reaction resulting in significant pathology at sites of inflammation.

Individuals who are immunocompromised or immunosuppressed have a reduced ability to mount cellular immune responses to fight infections and other diseases. This may be caused by certain diseases or conditions, such as AIDS, cancer, diabetes, malnutrition, and certain genetic disorders. These disease conditions cause loss of Th1 immunity and are characterized by lower Th1/Th2 balance. Loss of cellular immune function may also be caused by certain medicines or treatments, such as anticancer drugs, radiation therapy, and stem cell or organ transplant.

The effective simulated innate immune response resulting from the immunotherapy described herein can limit the course of virus infection by the early production of type II interferon (IFN-gamma) and support release of type I/III interferons (IFN) and subsequent switch to type II IFN (IFN-gamma) production as the immune response matures. Successful viral infections can become established due, in large part, to delayed innate immune responses, especially in the elderly, allowing rapid viral propagation which eventually can result in tissue destruction and dysregulated immune responses which can correlate with severe morbidity and mortality.

The immunotherapy regimen described herein can generate a ready pool of de-novo primed cellular immune memory cells that can respond rapidly to viral infection by producing IFN-gamma. IFN-gamma creates an anti-viral state that prevents viral infection of normal cells and prevents accumulation of high viral burden upon infection by the virus.

The de novo generation of non-exhausted Th1/CTL memory immune cells can modify the character of immune environment in the susceptible individual by providing a Th1 re-balancing. This can be accomplished through the creation of a high titer of polyclonal, allo-specific, non-exhausted, memory actions of activated allogeneic Th1-like cells (AlloStim®). Memory cells in the susceptible individuals can be readily activated non-specifically by environmental stimuli such as cytokines released by local cells in response to foreign pathogens. The allo-specific memory cells resulting from the priming are programmed to produce IFN-gamma upon activation. IFN-gamma can have a direct anti-viral effect on cells infected with virus and can also protect uninfected cells from infection. IFN-gamma can create the same anti-viral environment as innate release of Type I/III IFN does in an effective natural innate immune response.

The by-stander effect of this allo-specific Th1/CTL memory T-cell activation and IFN-gamma production can elicit protective effects on cells, for example, cells in the respiratory tract. Rapid release of IFN-gamma can support an effective innate immune response to virus and condition the microenvironment for an in-situ vaccination leading to viral-specific immunity and memory specific for the invading virus.

IFN-gamma can cause the maturation of dendritic cells (DC) to IL-12+ DC1 which are the link between innate and adaptive immunity. DC1 can process viral antigens, migrate to draining lymph nodes and present antigen to T-cells that are responsible for viral-specific adaptive immune response.

The immunotherapy regimen described herein can modulate the immune system of elderly adults to increase titers of non-exhausted memory Th1 cells and this can also provide the potential benefit of restoring cellular immune function in general by re-balancing systemic Th1/Th2 immunity. The increase in Th1 immunity in the elderly can have a counter-regulatory effect on any pre-existing chronic inflammatory processes. Chronic inflammation in the elderly is correlated with higher susceptibility to diseases that have increased incidence in the elderly such as cancer, arthrosclerosis, and Alzheimer's disease. Thus, vaccination of elderly pursuant to the immunotherapy regimen described herein has the potential not only to provide protection from progression of viral infections, but also may serve to down-regulate the type of chronic inflammation that is associated with diseases of aging.

The frequency and severity of infectious diseases can increase with old age. Infections carry a substantial risk of illness, loss of independence, disability, and death in elderly persons. This can contribute to the socioeconomic burden associated with rising life expectancy. Age-related changes in the immune system can hamper successful prophylactic vaccination in this population, even if a vaccine for viral infection is available (as for influenza) or eventually developed for COVID19. Accordingly, vaccines tailored to the needs of the aging immune system are a high priority for development and represent an unmet medical need.

Approaches to protective vaccine development and treatment for viral infection seek either to: (a) identify viral antigenic motifs which elicit neutralizing antibodies to block viral entry or use immune plasma from recovered patients, putatively containing therapeutic antibodies; (b) identify viral peptides which associate with and display on HLA subtypes (MHC I) that can be recognized by CD8+ T-cells and combine with adjuvants which support cellular immunity; or, (c) develop compounds which interfere with key points in the virus life cycle. These approaches can all have their technical and immunological challenges. For example, neutralizing antibody vaccines can be defeated by viral mutations or conformational changes in the virus attachment protein and/or by viral propagation through syncytium formation.

Defective cellular immune responses in the aged population, the most vulnerable to progression of viral disease, may not have the immune capacity to respond to a protective vaccine even if one is successfully developed. Viral peptide vaccines must be processed and administered with adjuvant which supports expression on MHC I and MHC II to elicit effective cellular immune protective responses upon viral infection. However, even if titers of viral specific Th1/CTL are successfully elicited, most viral infected cells down-regulate MHC I molecules to evade immune elimination. Finally, anti-viral drugs that show effectiveness tend to slow down viral propagation, but do not provide memory to protect against re-exposure to the same or an emergent variant virus strain or to a novel virus. In addition, anti-viral drugs can provide selective pressure for emergence of resistant strains.

In one embodiment, immunotherapy regimens described herein can overcome these design barriers to provide universal protection against viral infections. In one embodiment, immunotherapy regimens can be directed at elderly individuals. The immunotherapy regimen can modulate the immune systems of healthy elderly adults in a manner that “primes” them to respond quickly to any viral infection, overcoming the late innate response and cellular immune suppression common in the elderly immune response to viral infection. A healthy, properly balanced immune system can resolve viral infections quickly before viremia is established. The rapid clearance of the virus can reduce the risk of transmission of the disease to others and prevent tissue damage, high morbidity and mortality associated with progression.

In some embodiments, the compositions used in the immunotherapy regimens described herein can include allogeneic living immune cells, where at least a portion are T-cells. In one embodiment, the T-cells can be effector/memory T-cells (CD45RO+, CD62L^{Lo}) of the Th1 phenotype (CD4+ T-cells that produce IFN-γ and not IL-4). The effector/memory Th1 cells can be activated at the time of formulation or introduction to a patient. The activation of the T-cells can be through cross-linking of CD3 and CD28 surface molecules on the T-cells. Other activation methods may also be used and are also within the scope of this description.

The allogeneic T-cells cells can express CD40L upon being activated and produce large amounts of inflammatory cytokines (such as IFN-γ, GM-CSF, and TNF-α). Activation of T-cells to generate emTh-1 can be performed as described, for example, in U.S. Pat. No. 7,435,592 and formulated for administration in buffer as described in U.S. Pat. No. 7,402,431. Both patents are incorporated herein by reference. Activated emTh-1 cells can be used in the compositions described herein. Such activated emTh-1 cells are available under the trademark AlloStim® from Mirror Biologics Inc. Other activated allogeneic T-cells or components thereof that exhibit functional characteristics similar to these cells may also be used and are also within the scope of this description.

The universal vaccine compositions will be described in the context of including activated emTh-1 cells such as AlloStim® but vaccine compositions containing cells prepared using other methods having similar functional characteristics are all within the scope of this description. Also, in the scope of the description of the vaccine compositions are non-living components of emTh-1 cells, such as IFN-gamma and CD40L molecules.

AlloStim® is an “off-the-shelf”, non-genetically manipulated, living immune cell therapy containing living Th1-like memory cells. The cells can be administered in an activated state in the immunotherapy regimen by attaching anti-CD3/anti-CD28 conjugated microbeads that cross-link the CD3 and CD28 cell surface moieties. In one embodiment, AlloStim® is available in a frozen dosage form that has a 12-month shelf life, making it possible to distribute inventory to clinics most anywhere in the world using dry shippers. Local liquid nitrogen storage capability may be required.

In one embodiment, AlloStim® is derived from naïve CD4+ T-cells purified from the blood of healthy donors and is differentiated and expanded ex-vivo. AlloStim® can be intentionally mismatched to the elderly individual, e.g., the host, and can be rapidly rejected by the host immune system within 24 h of administration. Accordingly, because the transferred cells are rapidly rejected, they do not act as effector cells to eliminate viral infected cells, rather they can serve as immunomodulators and as an adjuvant to promote a host-mediated immune response. AlloStim® can express high density CD40L and produce cytokines such as IFN-gamma, GM-CSF, and TNF-alpha. When the vaccine composition that includes AlloStim® is injected intradermally to the host, the combination of the alloantigens, cytokines and CD40L expression can create a vaccine to prime for development of a Th1/CTL allo-specific immune response by the host.

Rejection of the intradermally administered AlloStim® graft by the host immune system can result in a non-toxic host vs graft (HVG) reaction which serves as an adjuvant to support a host vs. pathogen (HVP) effect, equal in potency and efficacy as a graft vs tumor (GVT) effect that occurs after allogeneic stem cell transplant of a patient with cancer that occurs in the context of GVHD toxicity. The HVG effect is the “mirror” of the GVT effect and occurs in the context of a non-toxic host vs. graft (HVG) rejection of AlloStim® which is the “mirror” of graft vs host disease (GVHD). The combined HVP/HVG mechanism is similar to the “Mirror Effect” as described, for example, in U.S. Pat. No. 7,435,592 to Har-Noy.

In one embodiment, the AlloStim® in the vaccine compositions can modulate the immune system of elderly adults. This immune modulation can provide a readily available pool providing an immediate release of regulatory cytokines, such as IFN-gamma, GM-CSF, TNF-alpha which serve to activate NK cells, cause differentiation of DC cells (IL-12+DC1) and non-specifically activate memory T-cells. The release of IFN-gamma from the activated pool of host allo-specific Th1 memory cells and NK cells can serve to protect uninfected cells from viral entry.

The activated host allo-specific memory Th1/CTL can also cross react with foreign viral antigens to provide immediate T-cell response to remove viral infected cells. These new viral-specific memory cells can both protect against re-infection from the same virus and can serve as an additional pool of non-specific memory cells that would be activated upon any variant or new viral encounter.

In some embodiments, the immunotherapy regimen can include the use of the universal vaccine. The universal vaccine composition may be administered via all the routes conventionally used including the parenteral, intradermal, intramuscular, subcutaneous, intranodal or mucosal routes. In one embodiment, the universal vaccine can be administered as one or more intradermal doses. The vaccine composition may be administered by the same route or different routes at each administration. In one embodiment, at least three intradermal doses are administered to a host. In one embodiment, at least four intradermal doses are administered to a host. In one embodiment, at least three intradermal doses are administered to a host.

The allogeneic emTh-1 cells in the universal vaccine can be derived from a deliberately HLA-mismatched donor. In one embodiment, the dosage in a universal vaccine composition is between about 1×10⁶ and about 1×10⁷ cells. In one embodiment, the dosage in a vaccine composition is at least about 1×10⁶ cells for intradermal route. In one embodiment, the dosage in the vaccine composition is about 0.5 mL of 1×10⁷ cells/mL composition. Dosages of outside this range that can primarily generate the desired immune response are also within the scope of this description.

Intradermal injections of the composition can “prime” a patient to become immune to the alloantigen in the vaccine composition. Multiple intradermal injections can increase the number of Th1 memory cells specific for the alloantigens in the circulation of the host, which in turn changes the Th1/Th2 balance. In one embodiment, intradermal injection can include about 1×10⁶ to 1×10⁷ cells of AlloStim®.

The intradermal dosing is preferably repeated multiple times to build up the number of circulating allo-specific Th1 memory cells. After the first dose, each subsequent dose of the composition may be administered at least 3 days after administration of the previous dose. In one embodiment, the vaccine composition may be administered intradermally and can be repeated about 3-6 times and about 3-10 days apart. In one embodiment, about 3 to 5 intradermal doses of the vaccine composition can be administered. In one embodiment, the intradermal doses of the vaccine composition can be administered about every 3-4 days. Administration of more than 3 to 5 doses and at intervals other than 3-4 days are also within the scope of this description. Additional doses of the therapeutic composition may be administered as needed.

In one embodiment, about 0.5 mL of AlloStim® at a concentration of about 1×10⁷ cells/mL are first administered intradermally. A second dose of about 0.5 mL of AlloStim® at a concentration of about 1×10⁷ cells/mL can be administered on about day 3/4. A third dose of about 0.5 mL of AlloStim® at a concentration of about 1×10⁷ cells/mL can be administered on about day 7. Optionally, a fourth dose of about 0.5 mL of AlloStim® at a concentration of about 1×10⁷ cells/mL may be administered on about day 10/11; and further optionally a fifth dose of about 0.5 mL of AlloStim® at a concentration of about 1×10⁷ cells/mL may be administered on day 14/15. Additional doses after a fifth dose may also be administered. The doses may be administered intradermally in the same location or a different location.

In some embodiments, the regimen is administered to elderly individuals. In one embodiment, elderly individuals can be about 50 years of age or over, or about 60 years of age or over, or about 65 years of age or over, or about 70 years of age or over, or about 75 years of age or over, or about 80 years of age or over, or about 85 years of age or over, or about 90 years of age or over.

When an individual primed by administration of the universal vaccine by a regimen described acquires a viral infection, additional doses of the universal vaccine may be administered. These additional doses may be administered intradermally and/or intravenously and/or as an inhaled composition of alloantigens. To activate memory cells generated by the universal vaccine regimen, an intravenous infusion of the AlloStim® cells alone can be administered. The method may also include administering the composition intravenously to cause the activation of host immune cells (both innate and adaptive) and their extravasation to sites of inflammation. In one embodiment, one or more intravenous doses of the composition of AlloStim® cells may include about 1×10⁷ to about 1×10⁹ cells, or about 5×10⁷ to about 1×10⁸ cells. The intravenous infusions may be repeated several times, for example, on a monthly basis.

The immunotherapy regimen with the universal vaccine can be effective against a variety of viral pathogens. Viral pathogens can include influenza virus A, influenza virus B, SARS, MERS, and/or coronavirus. In one embodiment, the universal vaccine can be effective against the coronavirus, e.g., COVID19.

In some embodiments, the present description can include co-injection with viral antigens, such as viral nuclear proteins. The administration of the immunotherapy regimen can result in in-situ generated antigens in the lungs of the individuals upon viral infection. The individual's immune response can then generate Th1 memory cells against the in-situ generated viral antigens to clear the viral load present and/or prevent reinfection or future infections from the same virus.

### EXAMPLE

The purpose of this example was to evaluate whether the immunomodulatory mechanism of AlloStim® can prime the immune systems of healthy elderly adults to rapidly respond to pathogen infection and counter-regulate chronic inflammation correlated with diseases of aging.

Indication: Universal vaccine for healthy elderly adults to prevent progression of viral infections and counter-regulate chronic inflammation correlated with diseases of aging such as atherosclerosis and Alzheimer's disease.

### Primary Objective(s):

Safety: defined by frequency of vaccine-related reactogenicity events during acute monitoring.

Efficacy: determined by longitudinal analysis of peripheral blood polyclonal Th1/Th2 ratio; peripheral blood allo-specific Th1 memory cell titer; and peripheral blood cytokine and gene expression response to ex-vivo viral challenge.

### Investigational Products, Dose, and Route of Administration:

AlloStim®: AlloStim® is derived from the blood of healthy, screened donors. Donors are tested for blood borne diseases (HIV I/II, HTLV I/II, HBV, HCV, EBV, CMV, Zika, Chagas and syphilis) and for any lifestyle or travel habits which might cause a risk for transmission of disease through the blood. Whole blood is collected from known donors that meet eligibility criteria and pass donor testing requirements per 21 CFR 1271 at FDA registered tissue processing center in Phoenix, AZ USA or from screened anonymous donors from a licensed blood bank in compliance with 21 CFR 600. Whole blood is processed in a class 100 glove box using aseptic technique to purify the buffy coat component. The buffy coat is processed into a frozen intermediate product called T-Stim. To produce T-Stim, CD4+ naïve cells are purified from the source buffy coat and cultured in bioreactors for 9 days, adding anti-CD3/anti-CD28 coated microbeads at an approximate 1:1 bead:cell ratio on days 0, 3 and 6. Over a 9-day process, the purified CD4 cells expand approximately 50-fold and differentiate from naïve CD4 cells to activated Th1 cell phenotype to become T-Stim. The intermediate T-Stim product is debeaded, washed and aliquoted into bags with cryogenic protective media and stored frozen in liquid nitrogen. T-Stim is stable in liquid nitrogen for at least 3 years. T-Stim is quarantined until proven sterile and low endotoxin by validated tests. The identity, function, and ability to differentiate into AlloStim® is tested for each batch prior to release. When AlloStim® is required for dosing in clinic, frozen T-Stim bags are removed from released frozen inventory, thawed, washed, and incubated for 4 h with CD3/CD28-conjugated microbeads. Over the approximate 4h incubation period, the T-Stim cells differentiate and are now referred to as AlloStim®. AlloStim® is defined as activated memory CD4+ Th1 memory cells with CD3/CD28-conjugated microbeads attached in an approximate 1:1 bead:cell ratio. The AlloStim® cells with the microbeads attached are suspended at about 1×10⁷ cells/mL and formulated in PlasmaLyteA supplemented with approximately 1% human serum albumin and approximately 2% DMSO. The AlloStim® suspensions are loaded into cryovials and shipped in liquid nitrogen dry shippers to the clinical site using a specialty courier service. The frozen dosage forms are stable for up to approximately 3 months in LN2.

### Study Participants

This study enrolled 40 healthy adults of which 20 were aged 65-74 years (mean=70.6 yo) and 20 were aged 75 years and older (mean=79.7 yo). Eligible volunteers were screened to be in general good health by physical exam and with complete blood counts, liver function and kidney function and inflammation marker clinical laboratory tests within acceptable normal reference ranges. Volunteers were excluded if they had: a history of autoimmune disease; were HIV positive; a history of organ transplant or tissue allograft; or, any uncontrolled concurrent serious medical or psychiatric illness. SARS-COV-2 (COVID-19) vaccination was required prior to enrollment and influenza vaccination was allowed 28 days prior to first dose and 28 days after last dose. Influenza A (IAV) IgG (EIA) and Influenza B (IBV) IgG (EIA) was determined at baseline and at Day 336. Volunteers were screened for COVID-19 antigen during the duration of the study at regular office visits.

### Protocol

Volunteers were administered approximately 0.5 ml of AlloStim® by ID injection on day 0, day 3 or 4, day 7, day 10 or 11 and day 14. Peripheral blood samples (approximately 20 ml) were collected on days 0, 10, 28, 168 and 336 in EDTA lavender top tubes. Peripheral blood mononuclear cells (PBMC) were isolated from the peripheral blood by Ficoll-Hypaque density gradient centrifugation, washed and suspended at approximately 1×10⁷ cells/ml in approximately 90% fetal bovine serum (FBS) and approximately 10% dimethyl sulfoxide (DMSO) and stored in LN2 until evaluated for cellular immune function. Adverse events (AEs) were monitored in each volunteer throughout treatment during the 336-day evaluation period.

### Mechanism of Action

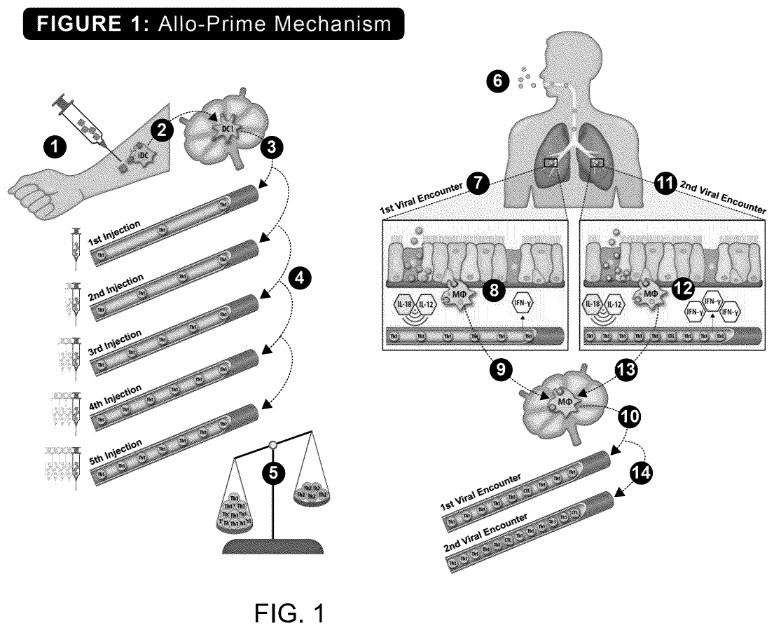

The hypothesized viral protection mechanism of Allo-Priming is shown in FIG. 1 which is a graphic representation of the method of action of this disclosure. The putative step-by-step mechanism of action of Allo-Priming is as follows: 1. Intradermal injection of AlloStim®. 2. The intentionally mis-matched allogeneic living AlloStim® cells are rejected by the host immune system and local immature DC engulf and process the allo-antigens in the context of Th1-steering danger signals. The iDC mature to IL-12+DC1, that express MHCI and CD80/86 co-stimulatory molecules, migrate to draining lymph node and present alloantigens to cognate T-cells. 3. Allo-specific Th1 cells enter the peripheral blood. 4. Upon each intradermal injection of AlloStim®, the titer of allo-specific Th1 cells in circulation increases. 5. The increases of circulating Th1 cells increases the Th1/Th2 balance. 6. Exposure to respiratory viruses in the environment initiate cellular immune responses. 7. Upon the first viral encounter, 8 alveolar macrophages engulf viral-infected cells in the respiratory tract which activates TLR7/8 and release of cytokines such as IL-18 and IL-12, which act to activate bystander Th1 cells which release IFN-γ, creating an anti-viral state. 9. The alveolar macrophages traffic to the draining lymph node and activate cognate viral-specific Th1/CTL. 10. Viral-specific Th1/CTL enter circulation further increasing the Th1/Th2 balance. 11. Upon a second viral encounter, 12. alveolar macrophages engulf and process viral infected cells and again cause bystander activation of Th1 cells with increased release of IFN-γ_. 13. The alveolar macrophages traffic to the draining lymph node and activate cognate Th1/CTL specific for the second virus. 14. The viral-specific Th1/CTL enter circulation, further increasing the Th1/Th2 balance. In this manner, a self-amplifying increase in cellular immune function mediated by the modulated Th1/Th2 balance results upon each environmental encounter with respiratory viruses.

Host rejection of multiple AlloStim® ID injections was predicted to elicit anti-alloantigen Th1 immunity, resulting in an increased titer of Th1 cells in circulation which is predicted to correct the elderly Th1/Th2 imbalance. Upon exposure to a respiratory viral infection, viral entry into bronchial endothelial cells or engulfment of viral infected cells by alveolar monocytes triggers internal toll-like receptors (TLR) and release of inflammatory cytokines, such as IL-12, IL-18 and IL-15. These cytokines are predicted to non-specifically activate the bystander allo-specific Th1 cells, causing their release IFN-γ into the microenvironment, creating an anti-viral state. The creation of an anti-viral state would maintain a low viral burden, providing sufficient time for an adaptive Th1/CTL viral-specific immune response that was capable of completely eliminating the offending virus (sterilizing immunity) to develop. After elimination of a first virus, the new viral-specific Th1/CTL cells now in circulation, added to the existing allo-specific Th1 cells created after Allo-Priming, would further increase the Th1/Th2 balance. Later infection with a different virus was predicted to result in an amplified IFN-γ release through bystander activation of both the resident expanded allo-specific Th1 cells and the Th1/CTL cells specific for the first virus. Upon each subsequent viral infection, bystander activation of memory allo-specific and viral-specific Th1 cells was predicted to cause increased release of IFN-γ and the expansion of the resident Th1 cells. In this manner, after remodeling the Th1/Th2 balance with Allo-Priming, subsequent environmental exposures to viruses was predicted to create a self-amplifying continuous strengthening of the cellular immune function over time, which would represent a type of trained immunity vaccine that could potentially provide broad protection from respiratory viral infections through cross-reactivity and through a heterologous immunity (HI) mechanism.

### Cellular Immune Function Assays

PBMC from longitudinal blood samples were analyzed for ability to produce IFN-γ by ELISPOT assay and supernatants from activated PBMC for ability to suppress viral lytic activity in a cytopathic effect (CPE) assay.

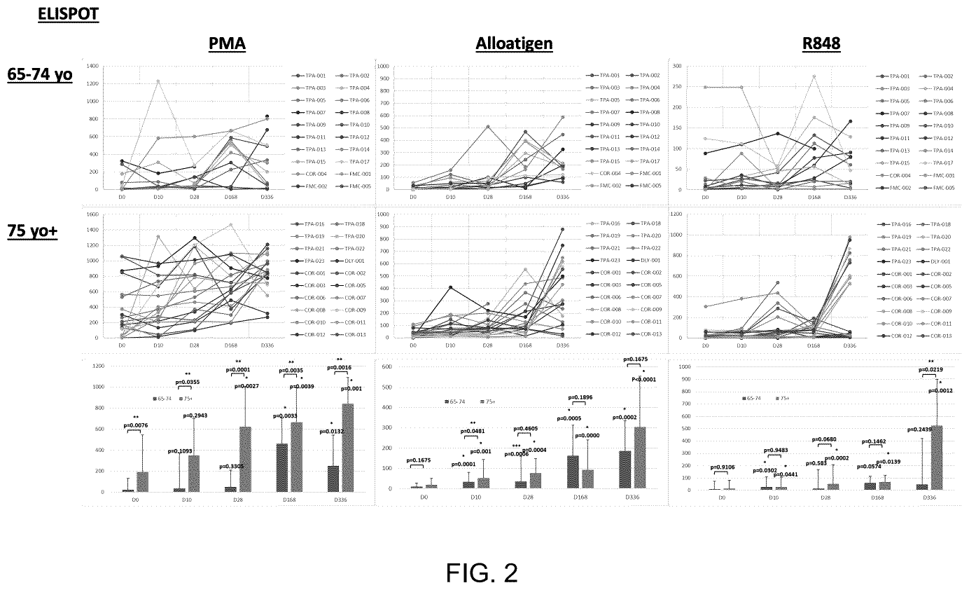

PBMC isolated from Day 0, 10, 28, 168 and 336 clinical research blood samples were incubated with cell activation cocktails: Phorbol myristate acetate (approximately 0.08 UM) with ionomycin (approximately 1.34 μM) (PMA) for polyclonal T-cell activation; R848 (Resiquimod) a TRL7/8 agonist (approximately 1 μg/mL) to simulate viral infection with a single stranded RNA (ssRNA) virus; and, a lysate from AlloStim® cells (approximately 250 μg/mL) to activate allo-specific T-cells. Activation times were approximately 24 h (PMA), approximately 72 h (AlloStim® lysate) and approximately 120 h for R848, all evaluated in triplicate in 96-well plates. (See FIG. 2)

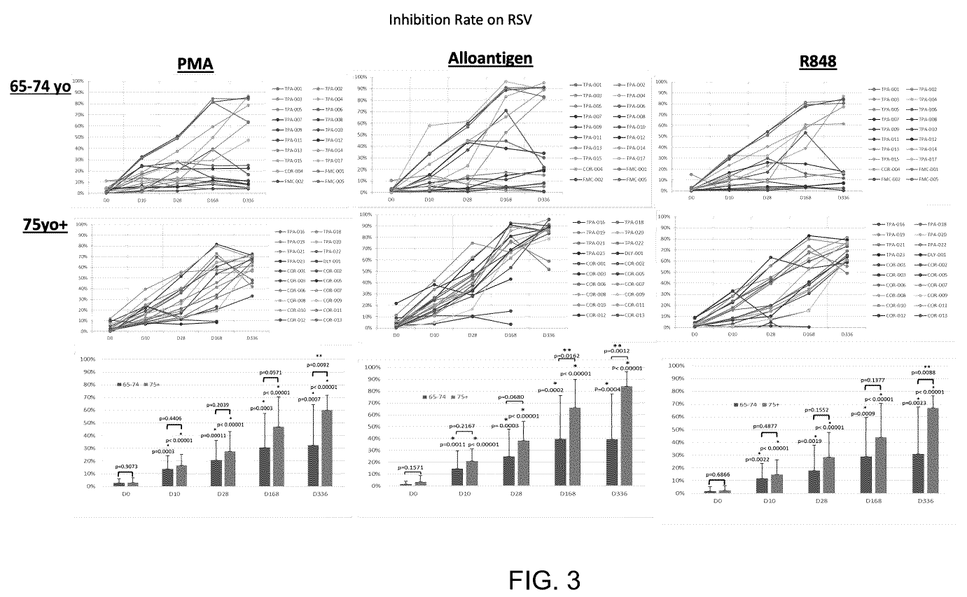

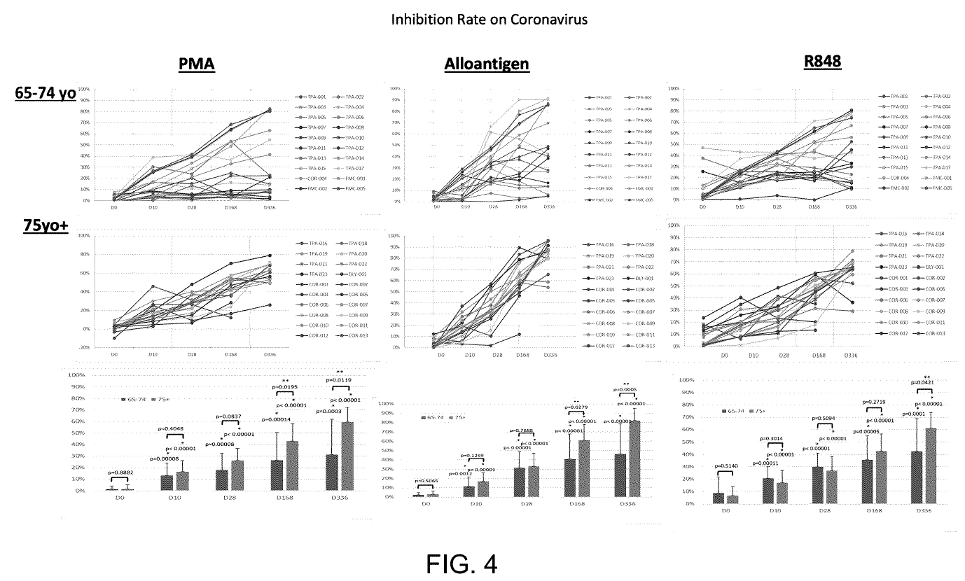

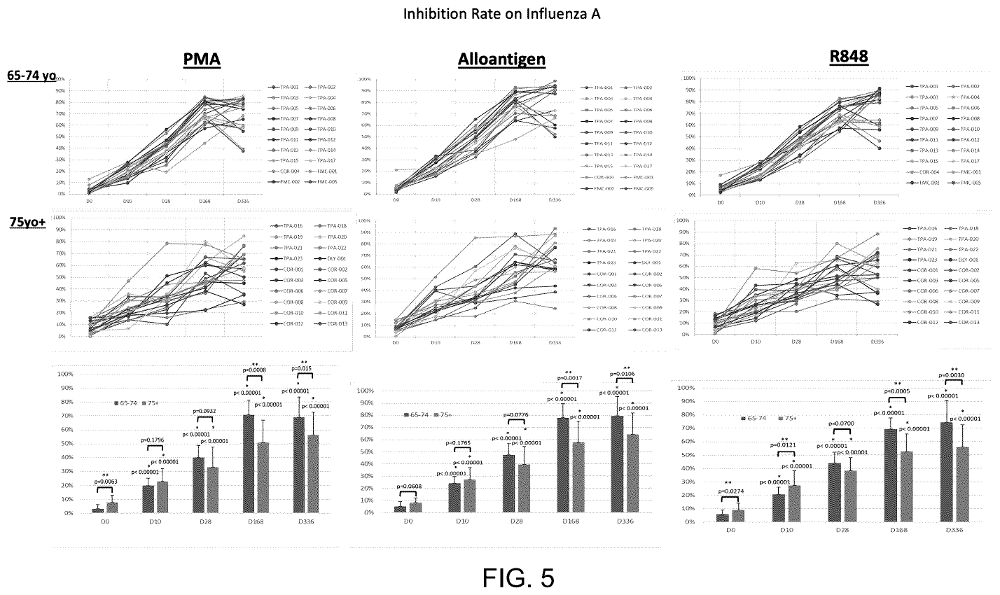

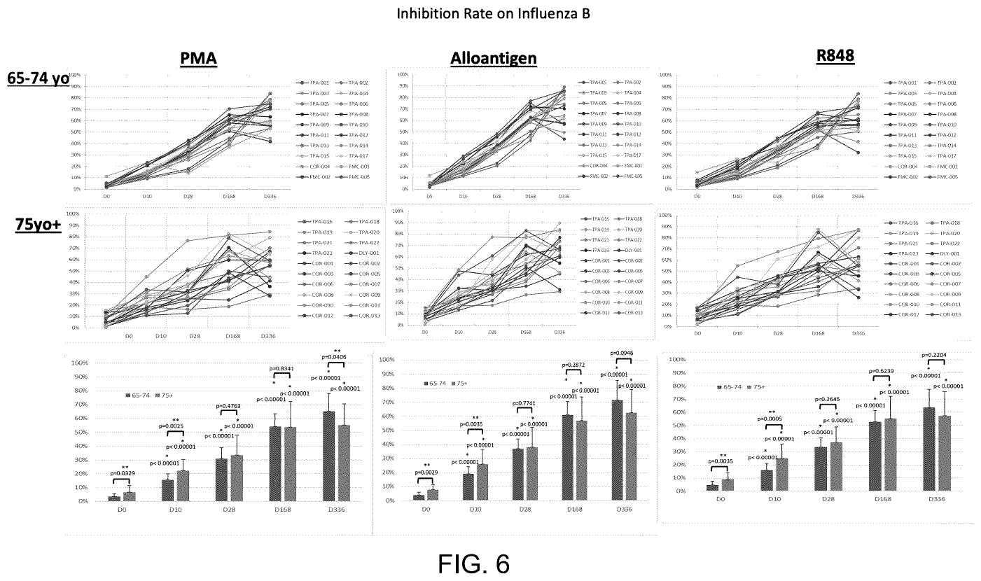

For the CPE assay, the supernatants from the activated PBMC were co-incubated with viral-infected cell line cultures each selected for their sensitivity to lysis by the selected live viruses: human respiratory syncytial virus (RSV) (ATCC, Cat #VR-26); Human coronavirus 229E (ATCC, Cat #VR-740); Influenza A virus (IAV) H1N1 (ATCC, Cat #VR-1894) and Influenza B virus (IVB) Victoria Lineage strain B/Florida/78/2015 (ATCC, Cat #VR-1931). Human MRC-5 lung fibroblast cells (ATCC, Cat #CCL-171) were the target cells used for RSV and coronavirus 229E cultures and Human HEK-293 kidney epithelial cells (ATCC, Cat #CRL-1573) were the target cells used for IVA and IVB cultures. (See FIG. 3 (RSV), FIG. 4 (Coronavirus), FIG. 5 (Influenza A), and FIG. 6 (Influenza B)).

The ELISPOT assay was performed using 96-well plates pre-coated with a monoclonal anti-human IFN-γ antibody, according to the manufacturer's instructions (R&D Systems #EL285). PBMC isolated from longitudinal clinical research blood samples from were incubated at approximately 1×106 cells/ml in separate wells of a 96-well plate for each activation cocktail. Secreted IFN-γ was captured on a Polyvinylidene difluoride (PVDF) membrane, detected by another anti-human IFN-γ antibody, and visualized with alkaline phosphatase and 5-bromo-4-chloro-3-indolyl-phosphate/nitro blue tetrazolium (BCIP/NBT) and analyzed using a Cytation 7 (Agilent) image analyzer and Gen5 software.

In the CPE assay, anti-viral activity was measured based on the ability of supernatants from activated longitudinal PBMC samples to inhibit virus-induced cytopathology in infected cell lines. Cell lysis was determined using a cellular adenosine triphosphate (ATP) detection reagent (Promega) and the luminescence intensity (LI) was measured using the Cytation 7 image analyzer. The viral infectivity and the corresponding tissue culture infective doses (TCID) were determined by serially diluting virus preparations and applying them to target cell lines for the specified exposure period. The virus dilution that produced a cytotoxic effect of approximately >80% of the cultured cells (TCID80) was calculated and used for all subsequent studies. Wells with and without virus (media alone) were used as controls. The percent inhibition of the lytic activity of each virus was normalized to approximately 100% corresponding to the LI in the media alone control and with approximately 0% corresponding to the LI in the virus alone control. As a functional positive control, known doses of recombinant IFN-γ were added to some virus-containing wells to confirm whether a linear viral suppression dose-response could be observed All wells were analyzed in triplicate.

### Flow Cytometry Analysis

PBMC from individual volunteer blood samples were stained using the following antibody panel: CD3-PE (BioLegend #300441), CD4-PerCP-Cy5.5 (BD Biosciences #560650), CD8-APC (BD Biosciences #566852), CD45RA-Pacific Blue (BioLegend #304123), and CD45RO-PE-Cy7 (BD Biosciences #560608). Samples were acquired using BD FACSLyric flow cytometer with BD FACSuite software (BD Biosciences). FACS files were analyzed using FlowJo software (BD Biosciences).

### 3. Results

### IFN-γ+ Th1 Cell Titers

To investigate whether Allo-Priming could increase the titer of circulating IFN-γ+ Th1 cells, PBMC isolated from clinical blood samples were analyzed for ability to produce IFN-γ after activation using an ELISPOT assay.

The potential for circulating T-cells to produce IFN-γ was determined by activation of the PBMC with PMA. The median number of PMA-activated T-cells producing IFN-γ was significantly greater in the 75 yo+ group compared to the 65-74 yo group at each time point. Within each age group, the median IFN-γ release was significantly increased at each time point compared to baseline in the 75 yo+ group. In the 65-74 yo group the median potential also increased at each timepoint, but only became significantly greater than baseline at days 168 and 336.

The number of allo-specific Th1 cells was determined by activation of PBMC with a lysate derived from the AlloStim® cells. Allo-specific Th1 cells were negligible in both age groups at baseline. After Allo-Prime dosing, the median number of allo-specific Th1 cells significantly increased in both age groups compared to baseline at each timepoint. The increase in allo-specific Th1 cells in the 75 yo+ group was significantly greater than the 65-74 yo group only at day 10, at all other time points the median number of allo-specific cells were not significantly different between the age groups.

To determine the capacity of monocytes in the PBMC to non-specifically activate bystander T-cells after TRL7/8 activation (simulating a ssRNA viral infection), PBMC were activated with R848. There was negligible bystander activation of IFN-γ+ cells after R848 activation in both age groups at baseline. There was no significant increase detected in the median number of IFN-γ producing cells after R848 activation compared to baseline in the 65-74 yo group at any timepoint except for day 10. In the 75 yo+ group, the median number of IFN-γ producing cells after R848 activation was significantly increased compared to baseline at each timepoint.

### Viral Lytic Activity Suppression

To determine whether supernatants from activated PBMC could suppress viral lytic activity in a CPE assay, supernatants collected from cultured PBMC activated with PMA, alloantigen or R848 were co-cultured with live virus cultures. The percent inhibition of viral lytic activity for coronavirus is shown in FIG. 3, RSV in FIG. 4, IAV in FIG. 5 and IBV in FIG. 6.

The viral suppressive activity of supernatants from all activation groups to all tested viruses at baseline were very low (approximately <20%). However, there was significantly better baseline suppression in the 75 yo+ age group against IAV and IBV compared to the 65-74 yo group at baseline, with no significant difference at baseline between the age groups for RSV or coronavirus. A consistent self-amplifying pattern of increased viral suppression over time was evident in all test groups, even after the Allo-Priming doses were completed.

In the coronavirus CPE cultures (FIG. 4), the viral suppression after PMA and alloantigen became significantly increased in the 75 yo+ group compared to the 65-74 yo group on day 168 and day 336 and at also significantly increased at day 336 after R848 activation. In the RSV CPE cultures (FIG. 3), the viral suppression in the 75 yo+ group compared to the 65-74 yo group was significantly higher at day 336 in all activation groups and significantly increased at day 168 and day 336, after alloantigen activation.

Whereas, viral suppression of coronavirus and RSV over time was significantly increased in the 75 yo+ group compared to the 65-74 yo group, a different pattern was observed in the influenza cultures. In the IAV CPE cultures (FIG. 5), the viral suppression in the 65-74 yo group compared to the 75 yo+ group was significantly higher at day 168 and day 336 in all activation conditions. In the IBV CPE cultures (FIG. 6), the viral suppression was also significantly higher in the 65-74 yo group compared to the 75 yo+ group after PMA activation at day 336. At all other earlier time points and activation conditions, viral suppression was amplified in time statistically equal between the age groups.

### Flow Cytometry

The changes in the circulating percentages of CD4+ CD45RO+ and CD8+ CD45RO+ memory T-cells was determined by flow cytometry at baseline and days 10, 28, 168 and 336 (see Table 2). No significant change in proportions of memory cells was found.

### SARS-CoV-2 Vaccine IgG Titers

7 of 19 (37%) volunteers in the 65-74 yo age group and 4/20 (20%) in the 75+yo age group presented with non-neutralizing SARS-COV-2 IgG titers at baseline after having been fully vaccinated. 5 of 7 (71%) of these low responders in the 65-74 yo group and 1 of 4 (25%) in the 75 yo+ group developed IgG neutralizing titers after Allo-Priming without additional COVID-19 vaccinations (see Table 3).

### COVID-19 Incidence

4 of the 40 (10%) volunteers were found to be positive for COVID-19 antigen at routine office visits during the 336-day observation period after Allo-Priming, with 3 of these 4 volunteers from the 65-74 yo age group and 1 of the 4 in the 75 yo+ age group (see Table 3). The findings were incidental and none of these COVID-19 antigen positive volunteers reported any symptoms at the time of detection.

### Adverse Events

A summary of reported adverse events (AE) are shown in Table 1. Most volunteers (77.5%) did not report any AE during the 336-day observation period. The most frequently reported AE was Grade 1 (mild) rash (10%) and Grade 1 non-COVID flu symptoms (7.5%). One serious adverse event (SAE) was reported in a 74 yo volunteer that was hospitalized with Grade 2 (moderate) upper respiratory pneumonia due to an unspecified organism (COVID-19, influenza A/B and MRSA were negative, rare gram-positive bacilli and cocci were present). This volunteer fully recovered and was discharged after 3 days of antibiotic and nebulizer therapy. One volunteer reported Grade 1 anemia. No other clinically significant changes in blood chemistries were reported.

### Discussion

Vaccines generally contain an antigen and an adjuvant. In Allo-Priming, the alloantigens expressed on the intentionally mismatched, living Th1 immune cells (AlloStim®), serve as the source of antigens and the activated AlloStim® cells' expression of high-density surface CD40L and secretion of IFN-γ serve as the key adjuvants that can steer the allo-specific immune response to a Th1-bias upon rejection by the host. The elderly generally present with an imbalance in circulating Th1/Th2 cell ratios, with loss of Th1 cells and IFN-γ.

A low amount of IFN-γ in response to respiratory viral infection is correlated with the age-related decline in vaccine efficacy and with increases in morbidity and mortality. Loss of innate cellular immune function in the elderly is associated with a decline in Th1 cells. In order to increase the titer of Th1 cells, repeated ID injections of AlloStim® cells were administered. The number of cells capable of releasing IFN-γ in response to an alloantigen pulse was analyzed by ELISPOT assay. The IFN-γ+ cells significantly increased after Allo-Priming and continued to increase over time after completion of dosing, providing support for the putative primary mechanism of action of Allo-Priming to enhance Th1 cell titers.

While the allo-specific Th1 cell titers were significantly increased over time, FACS analyses of T cell subpopulations did not show any major shifts in CD4+ or CD8+ CD45RO+ memory cells over the same timeframe (see Table 2). It is possible that a separate pool of activated memory cells that produce IFN-γ might have migrated to T-cell zones of peripheral lymph nodes or extravasated into tissues, resulting in an underestimation of the CD45RO+ memory cell fractions in the PBMC samples both in our study as well as prior studies with BCG vaccination.

The self-amplifying pattern of allo-specific Th1 cells without additional vaccinations suggests that these cells expanded over time in-vivo. Since Th1 memory cells can be activated without T-cell receptor (TCR) engagement through a bystander mechanism. The significant increases in allo-specific Th1 cells observed are believed to most likely be the result of Th1 cell expansion after bystander activation by cytokines released upon environmental encounters with viruses. Bystander T cell activation has been observed during viral-specific immune responses, and occurs preferentially among CD4+ memory T cells.

Evidence that the bystander activation mechanism of T-cells was intact and could be occurring in-vivo was demonstrated by the PBMC responses to R848 (Resiquimod). R848 is TLR 7/8 agonist. Engagement of these receptors in macrophages contained in the PBMC cultures mimics detection of single-stranded RNA (ssRNA) viral infections (e.g., coronaviruses, influenza viruses and RSV). TLR 7/8 signaling in macrophages can induce production of inflammatory cytokines, such as IL-12 and IL-18 that are capable of non-specifically activating bystander memory T-cells.

In an in-vivo setting, these monokines can potentiate Th1 immune responses to an ongoing infection, including in the elderly. The R848 activated T-cells analyzed by ELISPOT and the viral suppression of supernatants from R848-activated cells in the CPE assay support that TLR7/8 activation of monocytes could occur in the elderly and result in release of inflammatory cytokines that subsequently activate bystander Th1 memory cells to release IFN-γ. Bystander IFN-γ release can have a feed forward effect to activate and expand additional bystander T-cells, creating an inflammatory microenvironment which could replicate the early innate immune response that occurs in young individuals in response to respiratory viral infection.

Encounters with environmental pathogens can leave imprints on an immune system modulated by Allo-Priming which could then affect future immune responses to different viruses through a trained immunity mechanism. The modulated elderly immune system with restored cellular immune capacity could be remodeled through a similar mechanism as has been suggested by the ‘hygiene hypothesis’ which proposes that the building of a healthy immune system occurs through environmental exposures to pathogens.

The pattern of viral suppression in the CPE assays was shown to be significantly increased in the 65-74 yo group compared to the 75 yo+ group against IAV and IBV, while the suppression was significantly increased in the 75 yo+ group compared to the 65-74 yo group against RSV and coronavirus. These differences in anti-viral activity observed between the age groups to the different viruses could be related to resident trained immunity histories which might have resulted in a differential release of anti-viral cytokines upon activation at the different time points. A trained immunity effect might be more evident in the 65 yo-74 yo group against IAV/IBV due to better responsiveness to influenza vaccination in this group as compared to the 75 yo+ group.

Memory Th1 cells resulting from a primary viral infection can have a beneficial effect on subsequent infection with an unrelated virus through HI mechanisms. Training of innate immune cells, such as monocytes, macrophages and/or natural killer (NK) cells, by infection or vaccination, can enhance the immune responses against a new pathogen. For example, trained immunity of alveolar macrophages after viral infection in the lungs is known to support HI responses to unrelated viruses.

The self-amplifying increases in cellular immune function and anti-viral protection found here to occur over time, without additional vaccinating doses, supports the hypothesis that Allo-Priming could potentially provide broad respiratory viral protection to the elderly through HI and trained immunity mechanisms. Broad protection against respiratory viruses can occur via HI responses against unrelated subsequent viral infections, including against viruses such as RSV, rhinovirus, coronavirus and influenza viruses.

HI broad protection against respiratory viral infections after Allo-Priming may occur through a similar mechanism as has been shown after vaccination with BCG. Vaccination with the live BCG vaccine has been shown provide HI protection against non-related infections and reduce the incidence of respiratory infections in the elderly. HI protection after BCG vaccination occurs through a trained immunity mechanism that results in increased release of IFN-γ upon viral encounter. AlloStim® is a live vaccine that, like BCG, could lead to the long-term presence of memory Th1 cells and increased release of IFN-γ upon viral infection, which are associated with the HI and trained immunity responses to viral infection observed after BCG vaccination.

Clinical studies suggest that trained immunity can be utilized to enhance immune responses against infections and improve the efficiency of vaccinations in the elderly. BCG vaccination, for example, has been shown to increase titers of IgG. In this study, volunteers that presented with non-neutralizing SARS-COV-2 IgG titers at baseline, after having been fully vaccinated, developed IgG neutralizing titers after Allo-Priming without additional COVID-19 vaccinations (see Table 3). T-cell responses are positively correlated with the levels of neutralizing antibodies after vaccination. The increased cellular immune function demonstrated in the ELISPOT and CPE assays and the increase in COVID-19 IgG titers suggests that Allo-Priming may also have an effect on vaccine responsiveness in the elderly.

In 2023, adults aged 65 and older in the United States were significantly impacted by COVID-19. They accounted for approximately 62.9% of COVID-19 hospitalizations, 61.3% of intensive care unit admissions, and 87.9% of in-hospital deaths. During the 336-day observation period in this study, 10% of the volunteers tested positive for COVID-19 antigen on routine clinic visits without exhibiting any symptoms. An additional 2 volunteers presented to the clinic with mild non-COVID-19 flu-like symptoms. One volunteer was hospitalized for respiratory symptoms over the observation period and fully recovered. These real-life observations support that the enhanced cellular immune function that occurs after Allo-Priming may provide broad anti-viral protection against serious infection in this population.

The presumptive HI mechanism of Allo-Priming would have the advantage of potentially reducing the need for multivalent vaccines or advanced knowledge of the antigenic structure of circulating viruses in order to control community acquired viral infections. A vaccine with a HI mechanism would also potentially eliminate the need for frequent re-formulation of vaccines in order to maintain protection against viral mutant strains and could also provide protection from a future, currently unknown, pandemic viral pathogen.

This Phase I/II clinical trial has provided clinical proof-of-concept for the use of Allo-Priming as a strategy to possibly protect vulnerable elderly from severe symptoms from respiratory viral infection. The proof-of-concept data together with the excellent safety profile allows consideration for future clinical testing to evaluate the effects of Allo-Priming in high risk populations, such as assisted living facilities for the elderly, in order to determine in a randomized, controlled study whether Allo-Priming will prevent hospitalization and serious disease from community-acquired viral infections in the elderly.

TABLE 1
Adverse Events

| | | Incidence | |
|---|---|---|---|
| Event | Grade | (percent)) | |
| Rash | 1 | 4 | (10%) |
| Non-COVID Flu symptoms | 1 | 3 | (7.5%) |
| Back Pain | 1 | 2 | (5%) |
| Anemia | 1 | 2 | (5%) |
| Urinary Tract Infection | 1 | 1 | (2.5%) |
| Vasovagal Syndrome | 1 | 1 | (2.5%) |
| Epistaxis | 1 | 1 | (2.5%) |
| No AE Reported | | 31 | (77.5%) |

TABLE 2

| | | D0 | D10 | D28 | D168 | D336 |
|---|---|---|---|---|---|---|
| Age 65-74 | CD45RO⁺ % in CD4⁺ T cells (Avg ± SD) | 65.3 ± 15.5 | 63.0 ± 13.2 | 69.3 ± 12.0 | 56.7 ± 8.3 | 57.8 ± 13.5 |
| | CD45RO⁺ % in CD8⁺ T cells (Avg ± SD) | 39.2 ± 20.8 | 34.5 ± 14.8 | 40.1 ± 19.1 | 33.3 ± 12.6 | 36.7 ± 18.0 |
| Age >75 | CD45RO⁺ % in CD4⁺ T cells (Avg ± SD) | 62.9 ± 14.9 | 66.9 ± 15.7 | 69.0 ± 13.6 | 69.3 ± 16.6 | 63.5 ± 17.3 |
| | CD45RO⁺ % in CD8⁺ T cells (Avg ± SD) | 41.3 ± 16.9 | 43.8 ± 16.1 | 43.1 ± 20.5 | 43.8 ± 18.9 | 34.5 ± 2.6 |

TABLE 3

| COVID-19 Antigen | | | |
|---|---|---|---|
| Positive (Days after | # | | |
| AlloPrime) | Volunteers | 65-74 | 75+ |

| | | | | |
|---|---|---|---|---|
| <30 | days | 1 | 1 | 0 |
| 30-60 | days | 1 | 0 | 1 |
| 60-168 | days | 1 | 1 | 0 |
| 168-336 | days | 1 | 1 | 0 |

| | | | |
|---|---|---|---|
| Not infected | 36 | 17 | 19 |

What is claimed is:

1. A method of improving the immune response in an individual against a viral pathogen comprising:
administering allogeneic living immune cells where at least a portion are T-cells wherein the T-cells comprise effector/memory T-cells of a Th1 phenotype that produces IFN-γ and not IL-4.

2. The method of claim 1 wherein the allogeneic living immune cells are administered in at least three intradermal doses.

3. The method of claim 1 wherein the effector/memory T-cells express CD45RO+ and CD62LLo.

4. The method of claim 1 wherein the effector/memory Th1 cells are activated at formulation or introduction to the individual.

5. The method of claim 4 wherein the activation of the T-cells comprises cross-linking of CD3 and CD28 surface molecules on the T-cells.

6. The method of claim 1 wherein the T-cells cells can express CD40L upon being activated and produce inflammatory cytokines.

7. The method of claim 6 wherein the inflammatory cytokines comprise IFN-γ, GM-CSF, or TNF-α or combinations thereof.

8. The method of claim 1 wherein at least a portion of the effector/memory T-cells are derived from naïve CD4+ T-cells from a donor other than the individual.

9. The method of claim 2 wherein each of the doses of intradermal doses are administered at an interval of about 3 to 4 days.

10. The method of claim 1, wherein the invading pathogen is a virus.

11. The method of claim 1, wherein the invading pathogen is a coronavirus, RSV, Influenza A or Influenza B.

12. The method of claim 1, wherein the individual is over the age of 60.

13. The method of claim 1, wherein administration of the allogeneic living immune cells increases the level of IFN-gamma in the individual and/or the Th1 response after pathogen infection.

14. The method of claim 1, wherein the Th1/Th2 balance shifts toward Th1.

15. The method of claim 2, wherein a fourth, a fifth, or more intradermal doses are administered to the individual.

16. The method of claim 1 wherein the allogeneic living immune cells are administered with selected viral antigens.

17. A method of modulating the immune response in an individual at least 60 years of age comprising administering allogeneic living immune cells where at least a portion are T-cells wherein the T-cells comprise effector/memory T-cells of a Th1 phenotype that increases the Th1/Th2 ratio.

18. The method of claim 17 wherein the allogeneic living immune cells are administered in at least three intradermal doses.

19. The method of claim 17 wherein the effector/memory T-cells express CD45RO+ and CD62LLo.

20. The method of claim 17 wherein the effector/memory Th1 cells are activated at formulation or introduction to the individual.

21. The method of claim 20 wherein the activation of the T-cells comprises cross-linking of CD3 and CD28 surface molecules on the T-cells.

22. The method of claim 17 wherein the T-cells cells can express CD40L upon being activated and produce inflammatory cytokines.

23. The method of claim 22 wherein the inflammatory cytokines comprise IFN-γ, GM-CSF, or TNF-α or combinations thereof.

24. The method of claim 17 wherein at least a portion of the effector/memory T-cells are derived from naïve CD4+ T-cells from a donor other than the individual.

25. The method of claim 17 wherein each of the doses of intradermal doses are administered at an interval of about 3 to 4 days.

26. The method of claim 17, wherein administration of the allogeneic living immune cells increases the level of IFN-gamma in the individual and/or the Th1 response after pathogen infection.

27. The method of claim 18, wherein a fourth, a fifth, or more intradermal doses are administered to the individual.

28. The method of claim 17 wherein the allogeneic living immune cells are administered with selected viral antigens.

29. A method of increasing vaccine responsiveness in an individual fully vaccinated against at least one viral infection without additional doses of a vaccine for the at least one viral infection and comprising administering one or more doses of a vaccine composition to the fully vaccinated individual, the vaccine composition comprising allogeneic effector/memory Th-1 cells derived from a deliberately HLA-mismatched donor to the individual.

30. The method of claim 29 and further comprising administering at least three doses of the vaccine composition.
